(19) United States
(12) Patent Application Publication
(10) Pub. No.: US 2017/0273921 A1
(43) Pub. Date: Sep. 28, 2017

(54) 3,5,N-TRIHYDROXY-ALKANAMIDE OR 3,5,N-TRIHYDROXY-6-ALKENAMIDE DERIVATIVE ALONE OR IN COMBINATION WITH CHEMOTHERAPEUTIC AGENT FOR TREATING CANCER

(71) Applicants: Andrew Man Chung WO, Edgartown, MA (US); National Taiwan University, Taipei (TW)

(72) Inventor: Ching-Chow Chen, Taipei (TW)

(21) Appl. No.: 15/507,075

(22) PCT Filed: Aug. 14, 2015
(86) PCT No.: PCT/US2015/045208

§ 371 Date: Feb. 27, 2017

Related U.S. Application Data
(60) Provisional application No. 62/042,990, filed on Aug. 28, 2014.

(51) Int. Cl. A61K 31/16; A61K 31/22; C07C 259/06; A61K 31/4418; C07D 239/42; A61K 31/505; A61K 31/402

(52) U.S. Cl. CPC A61K 31/16; A61K 31/505; A61K 31/22; A61K 31/402; A61K 31/4418; C07D 239/42; C07C 259/06

(57) ABSTRACT

The present invention provides a novel method for treating cancer that comprises administering a compound represented by formula (I) and/or a chemotherapeutic agent to a subject. The present invention further provides a novel kit that comprises a compound represented by formula (I) and a chemotherapeutic agent for treating cancer.

### CROSS REFERENCE TO RELATED APPLICATION

The present application claims priority to U.S. Provisional Application No. 62/042,990, filed Aug. 28, 2014, entitled, “3,5, N-Trihydroxy-Alkanamide or 3,5,N-Trihydroxy-6-Alkanamide Derivative Alone or in a Combination with Chemotherapeutic Agent for Treating Cancer,” the entire contents of which is incorporated herein for all purposes by this reference.

### BACKGROUND OF THE INVENTION

### 1. Field of the Invention

The invention relates to a combination which comprises: (a) a 3,5,N-trihydroxy-alkanamide, 3,5,N-trihydroxy-6-alkenamide or a derivative thereof; and (b) one or more chemotherapeutic agents, for simultaneous, concurrent, separate or sequential use, especially for use in the treatment of cancers and cancer metastases. The invention also relates to a kit comprising such a combination and to a method of treating cancers and cancer metastases. The present invention further also relates to a commercial package or product comprising such a combination.

### 2. Description of Related Art

Colorectal cancer (CRC) is an important cancer with rising annual incidence worldwide, leading to significant morbidity and mortality. Most newly diagnosed CRC patients have localized diseases, for whom surgical resection with or without adjuvant chemotherapy provides chance of cure (1, 2). Despite aggressive treatment, a substantial proportion of patients suffer from metastatic CRC which includes metastatic diseases at diagnosis and metastatic recurrence. Metastatic CRC is a systemic disease essentially unresectable and remains incurable. Although new chemotherapeutic and molecular targeted agents in the past two decades prolong the survival from 12 to 24 months (3), the development of new drugs is crucial to further prolong the survival of metastatic CRC.

Statins reduce serum cholesterol levels by competitively inhibiting 3-hydroxy-3-methylglutaryl coenzyme A (HMG-CoA) reductase (HMGR), which is the rate-limiting enzyme in mevalonate and cholesterol biosynthesis. They are widely utilized to treat hypercholesterolemia and contribute to the decreased incidence of cardiovascular and cerebrovascular disorders (4). Statins have also shown promise related to cancer prevention in observational and preclinical studies and in certain aspects of randomized controlled studies (5).

We first demonstrated that statins with a carboxylic acid-containing long chain abrogate histone deacetylase (HDAC) activity to induce p21 expression through promoter histone-H3 acetylation and exert anti-proliferation and anti-tumor effect (6). Accordingly, statins can suppress tumor formation through inhibition of both HMGR and HDAC activities. Overexpression of HDACs inactivating tumor suppressor genes has been reported in cancer cells, supporting that HDACs are attractive drug discovery targets (7).

To further improve statins' HDAC inhibition, a drug potently inhibiting both HMGR and HDACs was sought. We designed and synthesized a series of 3,5,N-trihydroxy-alkanamide, 3,5,N-trihydroxy-6-alkenamide and their derivatives, that directly inhibited HMGR and class I and II HDACs, which suppressed cell viability in a variety of cancer cells but not normal cells (8). More specifically, PCT Patent Application No. W02014015235 reports 3,5,N-trihydroxy-alkanamide, 3,5,N-trihydroxy-6-alkenamide and their derivatives with HMGR and HDAC inhibitory activities.

### SUMMARY OF THE INVENTION

The inventors observed that a compound represented by formula (I) or a pharmaceutically acceptable salt thereof enhances the therapeutic effect of a chemotherapeutic agent in CRC as well as metastatic CRC such as liver metastasis or lung metastasis from CRC:

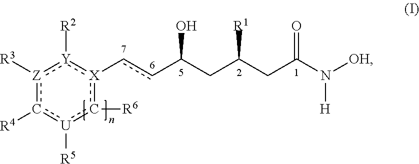

wherein:
n is 0 or 1;
[symbol] represents a single or double bond;
X is carbon;
Y, Z, and U are independently carbon or nitrogen; provided that, when Y is carbon and both Z and U are nitrogen, the bond between C6 and C7 is a double bond;
R¹, R², R³, R⁴, R⁵, and R⁶ are independently selected from the group consisting of null, H, C₁₋₆ alkyl, alkenyl, alkynyl, fluoroalkyl, chloroalkyl, bromoalkyl, iodoalkyl, perfluoroalkyl, aryl, heteroaryl, cycloalkyl, cycloalkenyl, aralkyl, aralkenyl, aralkynyl, heteroaralkyl, heteroaralkenyl, heteroaralkynyl, heterocyclyl, acyl, aminocarbonyl, amino, hydroxyl, alkoxy, acyloxy, silyloxy, amido, carbamoyl, and sulfonamido; and
R³ is optionally connected with R² or R⁴ to form carbocycle or heterocycle.

In one aspect of the present application, the compound represented by formula (I) is 3,5,N-trihydroxy-alkanamide, 3,5,N-trihydroxy-6-alkenamide or a derivative thereof. Preferably, the compound represented by formula (I) is (3R,5R)-7-{(1S,2S,6R,8S,8aR)-hexahydro-2,6-dimethyl-8-[2-methylbutyryloxy]naphthalenyl}-3,5-dihydroxy-N-hydroxyheptanamide (also referred to as JMF3086).

In one aspect of the present application, a method for treating cancers by administering a compound of formula (I) or a salt thereof and/or a chemotherapeutic agent is provided. In one embodiment of the present application, the compound of formula (I) and the chemotherapeutic agent are administered concurrently, separately or sequentially. In one embodiment of the present application, the chemotherapeutic agent is selected from the group consisting of an alkylating agent, a topoisomerase inhibitor, an antimetabolite agent, an anti-mitotic agent, and any combination thereof. Preferably, the alkylating agent can be cyclophosphamide, ifosfamide, cisplatin, carboplatin, oxaliplatin, or temozolomide, the topoisomerase inhibitor can be amonafide, amrubicin, amsacrine, campathecin, doxorubicin, epirubicin, etoposide, exatecan or irinotecan, the antimetabolite agent can be 5-fluorouracil, leucovorin, cladribine, capecitabine, mercaptopurine, pemetrexed, methotrexate, gemcitabine, hydroxyurea or cytarabine, and the anti-mitotic agent can be paclitaxel, docetaxel, vinblastine, vincristine, or vinorelbine. In another embodiment of the present application, the compound of formula (I) sensitizes the cancer cells to the chemotherapeutic agent.

In one embodiment of the present application, the cancer is caused by the proliferation of a neoplastic cell. In another embodiment of the present application, the cancer can be a solid tumor, a neoplasm, a carcinoma, a sarcoma, a leukemia, or a lymphoma, colorectal cancer (CRC), Hodgkin's disease, Non-Hodgkin's lymphomas, Ewing's sarcoma, multiple myeloma, Wilms' tumor, bone tumors, neuroblastoma, retinoblastoma, testicular cancer, thyroid cancer, prostate cancer, larynx cancer, cervical cancer, nasopharynx cancer, breast cancer, pancreatic cancer, head and neck cancer, esophageal cancer, small-cell lung cancer, non-small-cell lung cancer, brain cancer, melanoma and other skin cancers, a central nervous system (CNS) neoplasm, metastatic CRC, liver metastasis from CRC, or lung metastasis from CRC.

In one embodiment of the present application, at least one the additional cancer therapy that is selected from the group consisting of a chemotherapy, a radiation therapy, a therapeutic agent and any combination thereof is administered. Preferably, the therapeutic agent is bevacizumab, trastuzumab or cetuximab.

In some embodiments of the present application, the subject being treated in any of the treatment methods described herein can be a human or a non-human mammal.

In one aspect of the present application, a kit comprising a therapeutically effective amount of the compound represented by formula (I) or a pharmaceutically acceptable salt thereof and the chemotherapeutic agent is provided.

As one example of these 3,5,N-trihydroxy-alkanamide derivatives, JMF3086 enhances the therapeutic effects of oxaliplatin in AOM/DSS-induced CRC as well as liver metastasis or lung metastasis in mouse models.

As one example of these 3,5,N-trihydroxy-alkanamide derivatives, JMF3086 enhances the therapeutic effects of irinotecan in a liver metastasis mouse model as well as the therapeutic effects of Cisplatin in an orthotopic lung adenocarninoma mouse model.

### BRIEF DESCRIPTION OF THE DRAWINGS

FIGS. 1A and 1B show the effect of a 3,5,N-trihydroxy-alkanamide derivative, JMF3086, combined with oxaliplatin on colitis-induced colorectal cancer in vivo. Mice were intraperitoneally injected with AOM (12.5 mg/kg) and maintained for 7 days, then subjected to 3 cycles of DSS treatment (1 cycle representing 5 days of 3.5% DSS followed by 14 days of H₂O). After eight weeks, JMF3086 (25 mg/kg, 5 days/week) was orally administered to mice, oxaliplatin (5 mg/kg, once a week) was intraperitoneally administered to mice, and JMF3086 (25 mg/kg, 5 days/week and oxaliplatin (5 mg/kg, once a week) were administered to mice for additional two weeks. All mice were sacrificed at 10 weeks and colon segments were fixed by formalin. FIG. 1A shows schematic overview of test compounds administration. FIG. 1B shows gross pictures of terminal colons. Arrowhead shows macroscopic lesions. The number of tumors per mouse and the total tumor size (represented as the sum of the diameter of all the tumors) are graphed. Scale bars: 1 mm **P<0.01 versus vehicle.

FIGS. 2A to 2F show the effect of a 3,5,N-trihydroxy-alkanamide derivative, JMF3086, alone or combined with oxaliplatin or irinotecan on colorectal cancer metastasis to the liver or the lung in vivo, and on angiogenesis. FIG. 2A shows schematic overview of the experimental design (upper panel) for liver metastases of colorectal cancer. FIG. 2B shows the luciferase activity was detected by the IVIS imaging system after JMF3086 combined with oxaliplatin treatment for 2 weeks. FIG. 2C shows the luciferase activity detected by the IVIS imaging system after JMF3086 combined with irinotecan treatment for 2 weeks. FIG. 2D shows schematic overview of the experimental design (upper panel) for lung metastases of colorectal cancer. FIG. 2E shows gross pictures of lungs (scale bars: 5 mm) and their sections counterstained with H&E (scale bars: 250 μm), and quantitation of metastatic tumor nodules in mice. **P<0.01 versus vehicle. FIG. 2F shows the photographs of matrigel plugs retrieved from mice, and quantification of neovessel formation in matrigel plugs. RPMI medium was served as negative control. ##P<0.01 versus normoxia; **P<0.01 versus hypoxia.

FIGS. 3A and 3B show the effect of a 3,5,N-trihydroxy-alkanamide derivative, JMF3086, combined with cisplatin in an orthotopic lung adenocarninoma mouse model. FIG. 3A shows schematic overview of the experimental design (upper panel). FIG. 3B shows the luciferase activity detected by the IVIS imaging system after JMF3086 combined with cisplatin treatment for 2 weeks.

FIG. 4 shows the effect of 3,5,N-trihydroxy-alkanamide derivatives and the corresponding statins on cell viability of various human cancer cell lines.

FIGS. 5A to 5D show genome-wide analysis of target genes in 3,5,N-trihydroxy-alkanamide derivative-treated colorectal cancer cells. FIG. 5A shows schematic overview of working model was indicated (upper left panel). Ingenuity Pathways Analysis (IPA) analysis predicted that the genes associated with apoptosis were regulated by NR3C1. P=4.30E-37. FIG. 5B shows IPA analysis predicted that the inflammatory genes were regulated by NF-κB. The molecular network has a P-score (−log10 (p-value)) of 48. FIG. 5C shows that genes with two-fold down-regulation of H3K27-Ac in log-ratio were shown in green, while those with two-fold up-regulation were shown in red. FIG. 5D shows ChIP-qPCR was performed by immunoprecipitation with control rabbit IgG or anti-H3K27-Ac, anti-CBP, anti-HDAC1 and anti-HDAC3 antibody to detect their differential binding to the promoters of TNF-α, CD166, COX-II, BCL-2, TIMP3, and BMP2. Data were analyzed by the Q-PCR and plotted as percent (%) of input DNA. *P<0.05, **P<0.01 versus basal, two-sided Student t test.

### DETAILED DESCRIPTION OF THE INVENTION

The following specific examples are used to exemplify the present invention. A person of ordinary skills in the art can conceive the other advantages of the present invention, based on the disclosure of the specification of the present invention. The present invention can also be implemented or applied as described in different specific examples. It is possible to modify and or alter the examples for carrying out this invention without contravening its spirit and scope, for different aspects and applications.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

The term “cancer” used herein refers to any of many diseases characterized by the presence of cancerous tissue in a subject. As used herein, “cancerous tissue” refers to a tissue that comprises malignant neoplastic cells, exhibits an abnormal growth of cells and/or hyperproliferative cells. Cancerous tissue can be a primary malignant tumor, arising from a tissue or organ of origin, or it can be a metastatic malignant tumor, growing in a body tissue which was not the source of the original tumor.

The term “metastasis” refers to the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor.

The term “chemotherapeutic agent” used herein refers to an agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents include, for example, alkylating agents, such as cyclophosphamide, ifosfamide, cisplatin, carboplatin, oxaliplatin and temozolomide; topoisomerase inhibitors, such as amonafide, amrubicin, amsacrine, campathecin, doxorubicin, epirubicin, etoposide, exatecan and irinotecan; antimetabolite agents, such as 5-fluorouracil, leucovorin, cladribine, capecitabine, mercaptopurine, pemetrexed, methotrexate, gemcitabine, hydroxyurea and cytarabine; and anti-mitotic agents, such aspaclitaxel, docetaxel, vinblastine, vincristine and vinorelbine.

The term “cancer therapy” used herein refers to any method(s), composition(s), and/or agent(s) that can be used in the treatment of a cancer or one or more symptoms thereof. Cancer therapies include, for example, chemotherapy, radiation therapy, and targeted therapy. As used herein, “targeted therapy” refers to a means which acts directly against abnormal proteins in cancer cells. The targeted therapy may comprise, for example, administering a therapeutic agent such as bevacizumab, trastuzumab and cetuximab to a subject.

Many examples have been used to illustrate the present invention. The examples below should not be taken as a limit to the scope of the invention.

### EXAMPLES

### Example 1

### Animal Model of AOM/DSS-Induced CRC

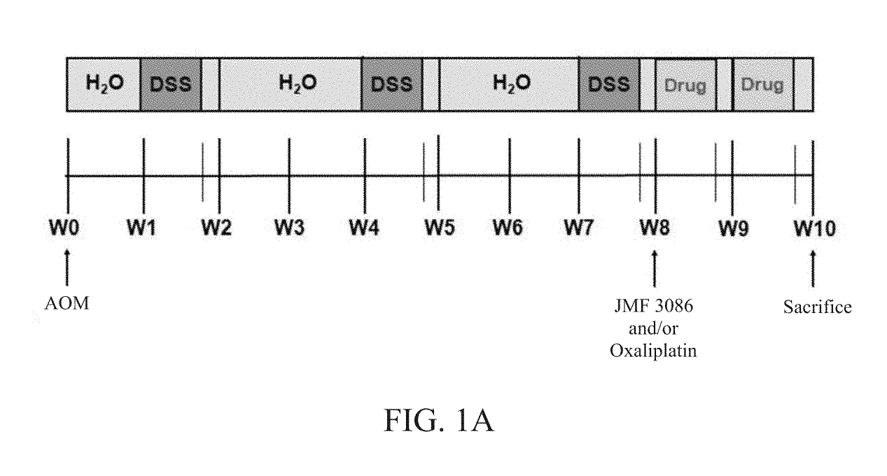

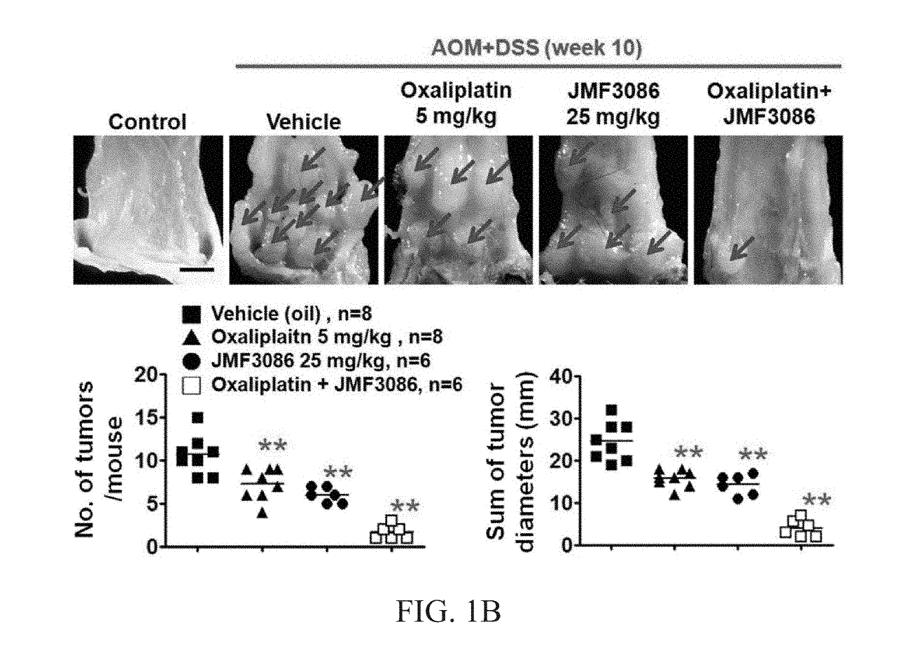

AOM in conjunction with the additional DSS inflammatory stimulus results in tumor development that is restricted to the colon in mice (11, 12). This model is widely utilized to recapitulate human colorectal cancer because it results in inflammation and ulceration of the entire colon, similar to what is observed in patients (12). CRC was induced in male C57BL/6 mice via the intraperitoneal injection of AOM (12.5 mg/kg) while the mice were maintained with a regular diet and drinking water for 7 days and then subjecting to 3 cycles of DSS treatment, with each cycle consisting of the administration of 3.5% DSS for 5 days followed by a 14-day recovery period with regular water (FIG. 1A). After 8 weeks, JMF3086 (25 mg/kg, 5 days/week) was orally administered to mice, oxaliplatin (5 mg/kg, once a week) was intraperitoneally administered to mice, and JMF3086 (25 mg/kg, 5 days/week) and oxaliplatin (5 mg/kg, once a week), irinotecan or 5-fluorouracil (5-FU) were administered to mice for additional two weeks. JMF3086 or oxaliplatin alone modestly reduced the number of tumors and sizes (FIG. 1B). However, combined treatment of JMF3086 and oxaliplatin significantly reduced CRC tumor growth (FIG. 1B). These results indicated that combined treatment enhanced the therapeutic effect of oxaliplatin on AOM/DSS-induced CRC.

### Example 2

### Animal Model for Experimental Colorectal Cancer Metastasis to the Liver and the Lung

Seven-week-old female NOD/SCID mice were obtained from the National Laboratory Animal Center. The mice were anesthetized by continuous flow of 2-3% isoflurane. To generate liver metastases derived from human colorectal cancer cells in mouse models, HT29-Luciferase expressing cells (1×10⁶) were suspended in 100 μl PBS and injected into the spleen of mice. After one-week recovery, the mice were randomized into vehicle and treatment group. Mice were oral administration of JMF3086 (5 days/week), intraperitoneally administered oxaliplatin, irinotecan or 5-FU (once a week) or a combination of oxaliplatin and JM3086, irinotecan and JM3086 or 5-FU and JM3086 for additional two weeks. Mice were then given endotoxon-free luciferse substrate and photographed by IVIS imaging system (Xenogen) once a week. Tumors in spleen and liver were collected and weighed after sacrificed.

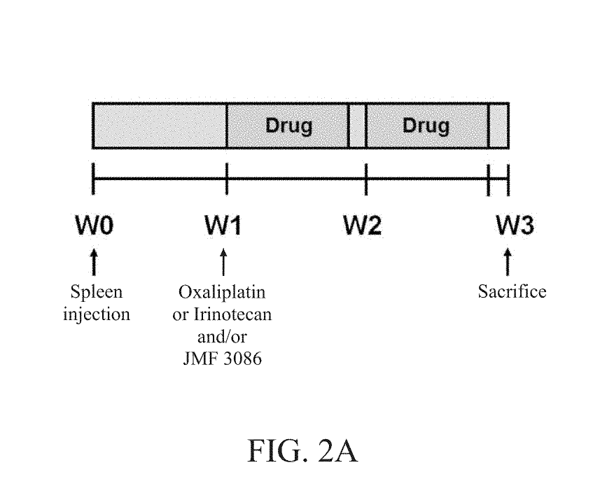

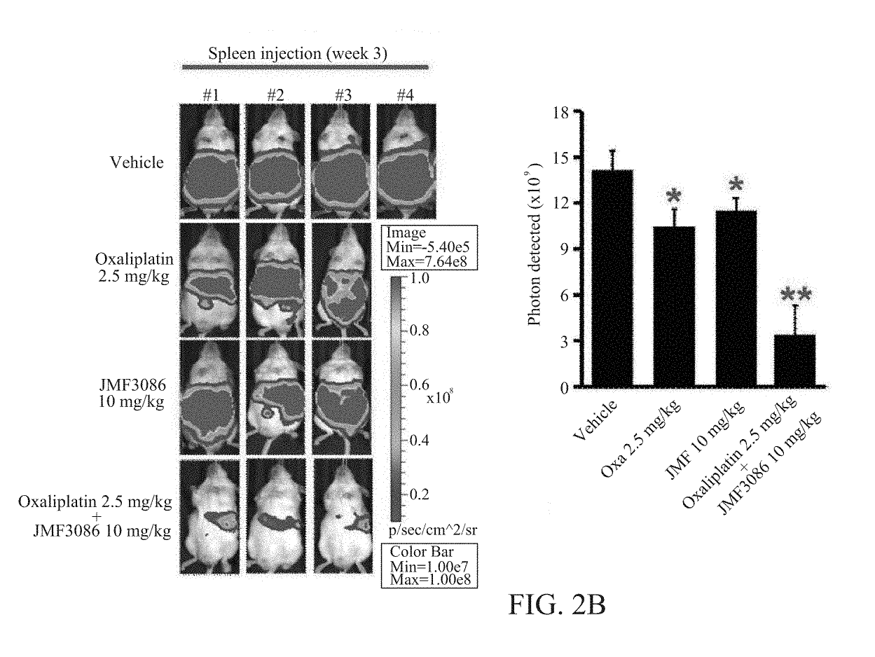

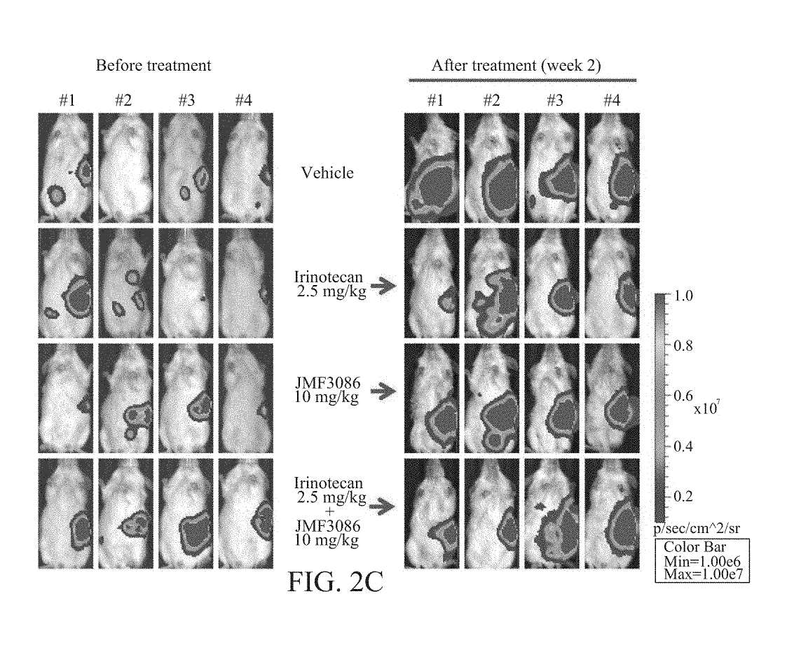

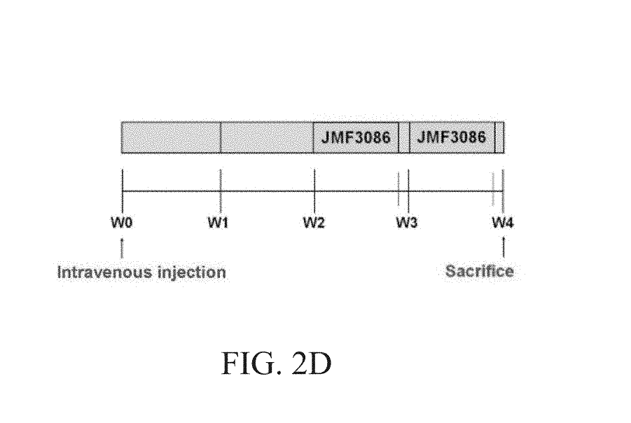

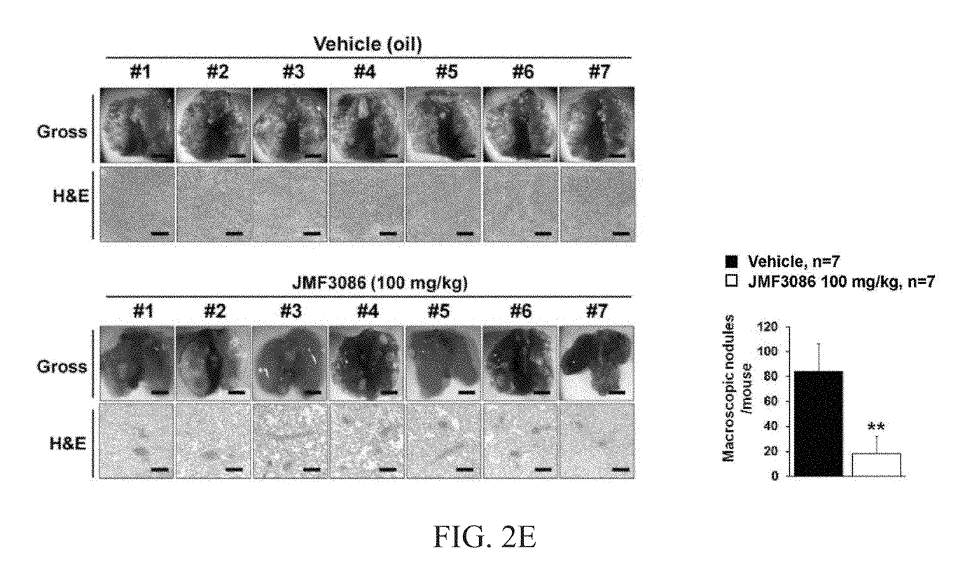

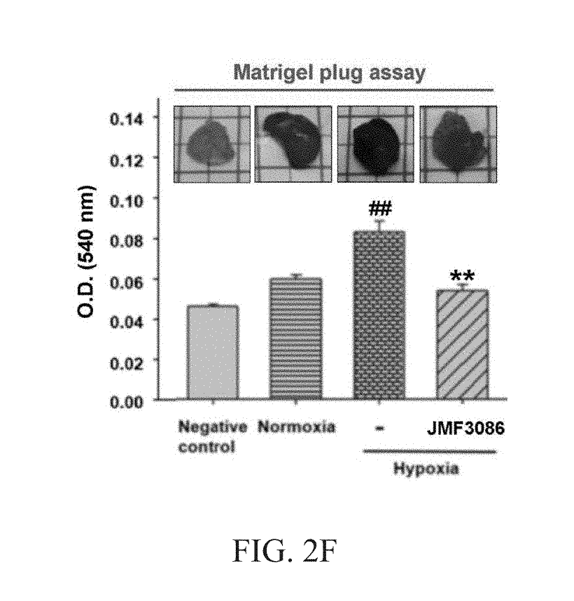

Metastasis is a major cause of death for CRC, and most CRC patients develop lung or liver metastasis (9, 10).To evaluate the anti-metastatic activity of JMF3086 and oxaliplatin or irinotecan combined treatment, HT29-Luciferase expressing cells were injected into the spleen of NOD/SCID mice and photographed by IVIS imaging system (FIG. 2A). A significant amount of tumor cells growing in the spleen and liver after one week injection was observed, and JMF3086 or oxaliplatin or irinotecan alone modestly inhibited the liver metastasis (FIGS. 2B and 2C). However, combined treatment of JMF3086 and oxaliplatin or irinotecan significantly reduced CRC metastasis to liver (FIGS. 2B and 2C).

Besides, to examine the anti-metastatic activity of JMF3086, colorectal cancer cells (HCT116) were intravenously injected into nude mice via the tail vein (FIG. 2D). JMF3086 (100 mg/kg) reduced lung tumor nodules by macroscopic observation (FIG. 2E), indicating its inhibition on lung metastasis.

Angiogenesis is an essential step of tumor growth and metastasis (13). To determining the effect of JMF3086 on angiogenesis, HCT116 cells treated with 30 μM JMF3086 for 20 hours were exposed to hypoxia for 4 hours, then conditional medium was collected and subcutaneous matrigel plug assay was conducted by injecting of 75 μl conditional medium mixed with 425 μl matrigel and 50 U heparin/ml into nude mice (5 mice/group). Mice were sacrificed and dissected after 14 days. Matrigel plug retrieved from mice were photographed. Quantification of neovessel formation in matrigel plugs were estimated using Drabkin reagent kit 525.

Conditioned medium from HCT116 cell sunder hypoxia induced angiogenesis by matrigel plug assay in mice, and this effect was inhibited by JMF3086 (FIG. 2F), indicating that JMF3086 had anti-angiogenesis effect.

### Example 3

### Development of Orthotopic Lung Cancer Models

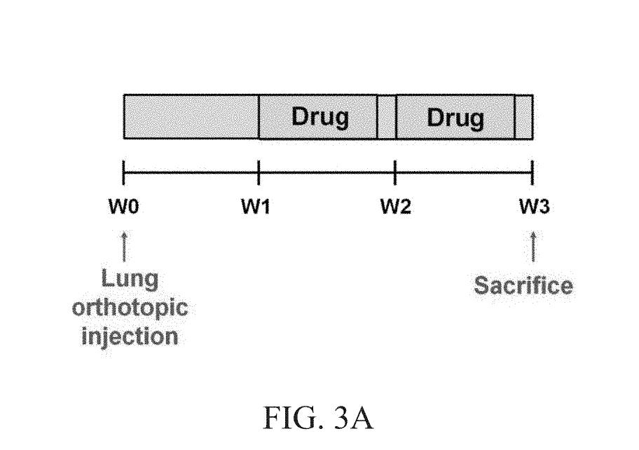

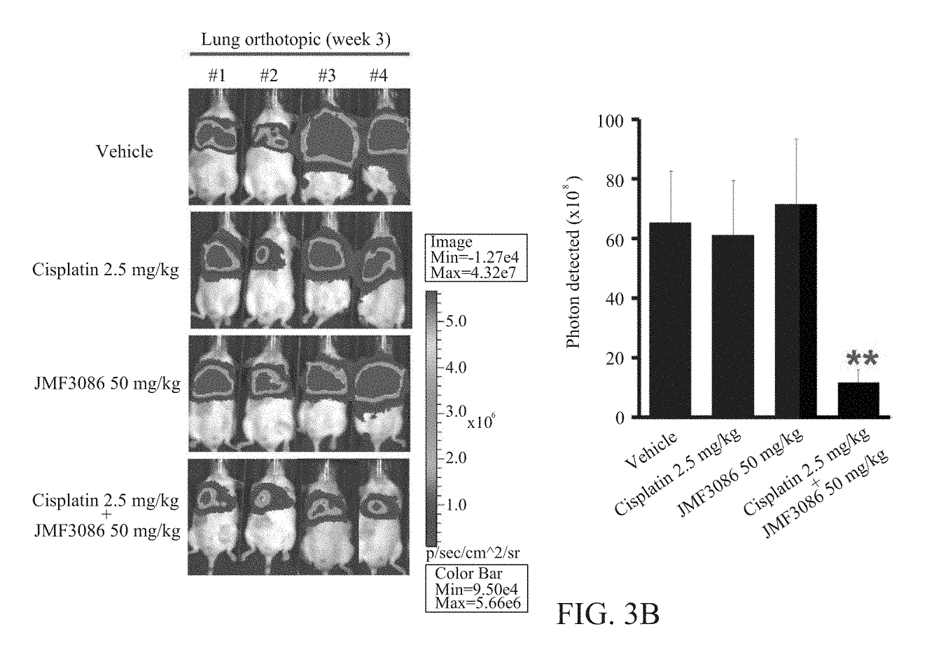

In this Example, we use A549 cells [non-small cell lung cancer cell line with KRAS mutations (EGFR wild-type)] to establish the orthotopic lung cancer models. Seven-week-old male NOD/SCID mice were anesthetized by continuous flow of 2-3% isoflurane. A549-Luc cells (2×10⁶) suspended in 40 μl of PBS were injected percutaneously into the right lung of the mice using 500 μl Gas-Tight syringes with 27G top winged infusion set. These mice were then given endotoxon-free luciferse substrate and photographed using IVIS-200 imaging system once a week. When lung tumor nodules were detected, the mice were oral administration of JMF3086 (5 days/week), intraperitoneally administered cisplatin (once a week) or a combination of JM3086 and cisplatin for additional two weeks (FIG. 3A). A significant amount of tumor cells growing in the lung after one week injection was observed, and JMF3086 or cisplatin alone ineffectively inhibited the lung cancer development (FIG. 3B). However, combined treatment of JMF3086 and cisplatin significantly reduced the tumor cells growth in the lung (FIG. 3B).

### Example 4

### Cell Proliferation Assay

Human ovarian cancer cells (Hela) and breast cancer cells (MD-MBA-231, MCF7) were seeded at 3000 cells/well in 96-well plates and maintained for 14-16 hours, then treated with JMF3086 or gemcitabine or both. After 72 hours, cells were washed with PBS then added medium containing MTT reagent (Sigma) at a final concentration of 0.5 mg/ml for 4 hours. Then, the medium was replaced with 200 μl of DMSO for 30 minutes, and using an ELISA reader (570 nm) to get the absorbance density values. The 50% of inhibition concentration (IC₅₀) of death cells was calculated.

### Example 5

### Effect of 3,5,N-trihydroxy-alkanamide Derivatives on Cell Viability

Effect of three 3,5,N-trihydroxy-alkanamide derivatives, JMF3086, (3R,5R)-7-{(1S,2S,6R,8S,8aR)-hexahydro-2,6-dimethyl-8-[2-dimethylbutyryloxy]naphthalenyl}-3,5-dihydroxy-N-hydroxyheptanamide (also referred to as JMF3171), and (3R,5R)-7-{[2-(4-fluoro-phenyl)-5isopropyl-3-phenyl-4-phenylcarbamoylpyrrol-1-yl]-3,5-dihydroxy-N-hydroxyheptanamide (also referred to as JMF3173) and the corresponding statins on the cell viability of various human cancer cell lines was determined by cell proliferation assay as described in Example 4. Specifically, cancer cells were treated with various concentrations of test compounds for 72 hours, and cell viability was measured by MTT assay. The half maximal inhibitory concentration (IC₅₀) values for individual cell line were calculated.

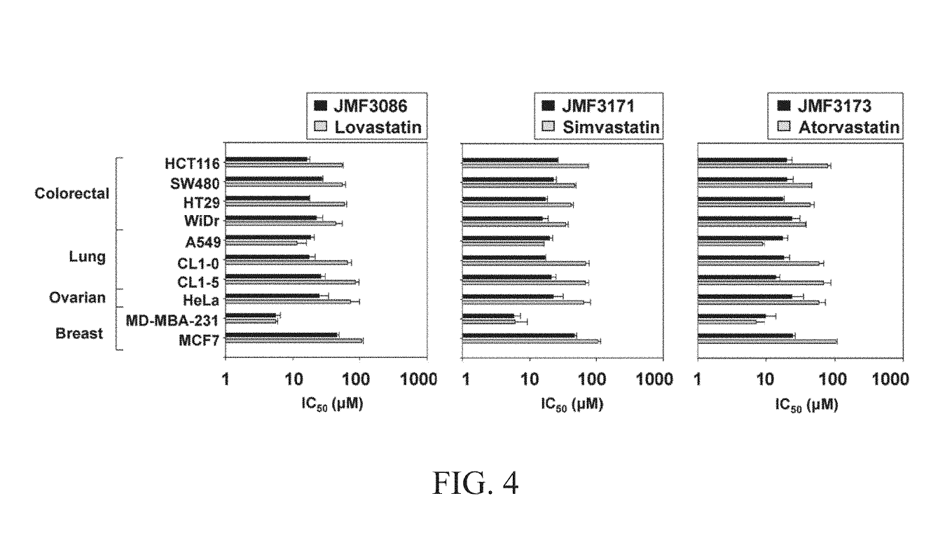

As shown in FIG. 4, the three 3,5,N-trihydroxy-alkanamide derivatives, JMF3086, JMF3171, and JMF3173, were cytotoxic in CRC and various cancer cell lines.

### Example 6

### Chromatin Immunoprecipitation (ChIP) and ChIP-on-Chip Assays

Colorectal cancer cells (HCT116) were treated with 30 μM JMF3086 for 24 hours, ChIP assays using control Rabbit IgG (sc-2027; Santa Cruz) or anti-H3K27-ac antibody (#4353; Cell Signaling) were performed as previously described (14).

ChIP-on-chip assays were performed in the Microarray and Gene Expression Analysis Core Facility of the National Yang-Ming University VGH Genome Research Center in Taiwan using the SurePrint G3 Human Promoter 1×1M Kit (Agilent Technologies) after DNA was further purified through phenol/chloroform/isoamyl alcohol extraction and ethanol precipitation. Genes with two-fold down-regulation or up-regulation of H3K27-Ac in log-ratio after JMF 3086 treatment were regarded as significance.

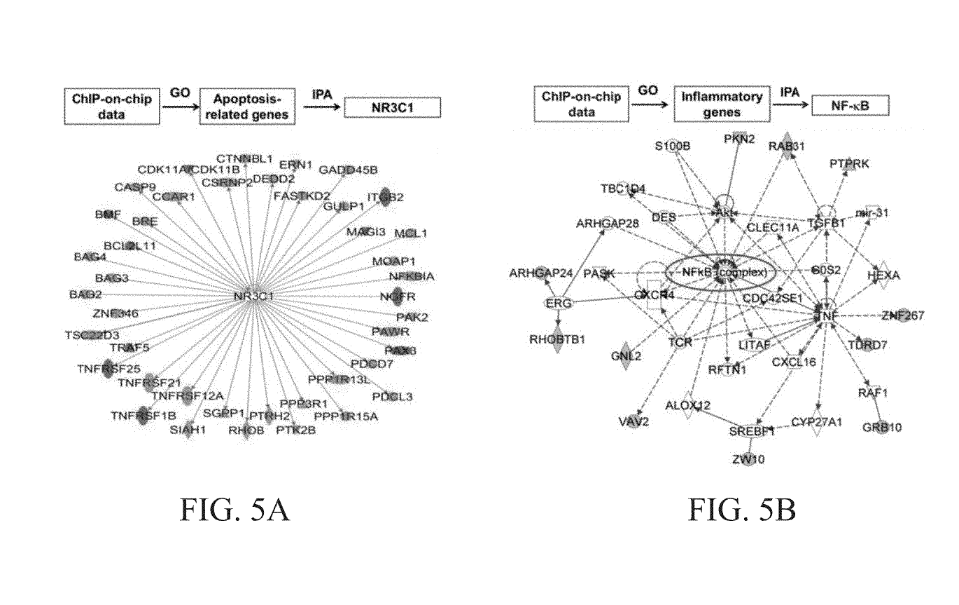

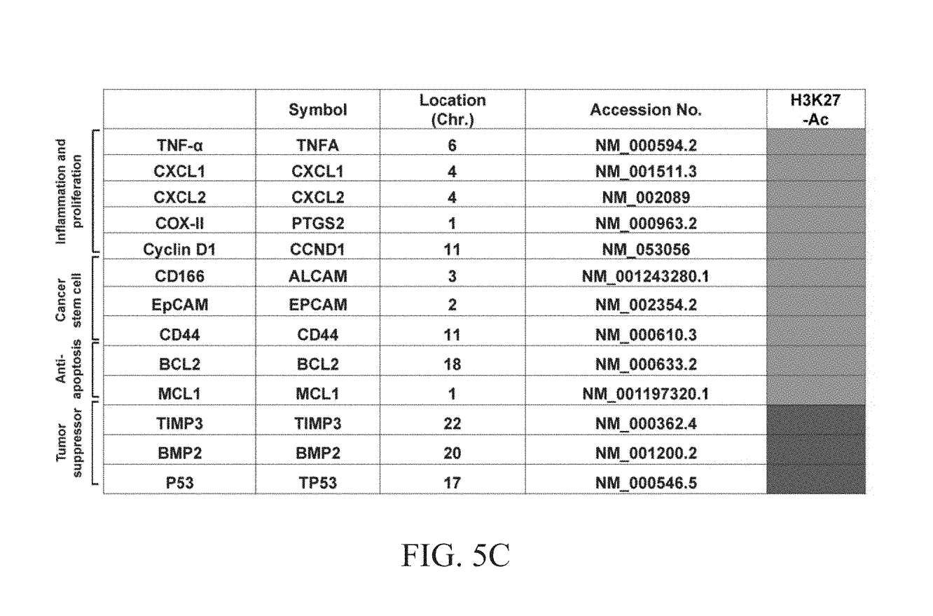

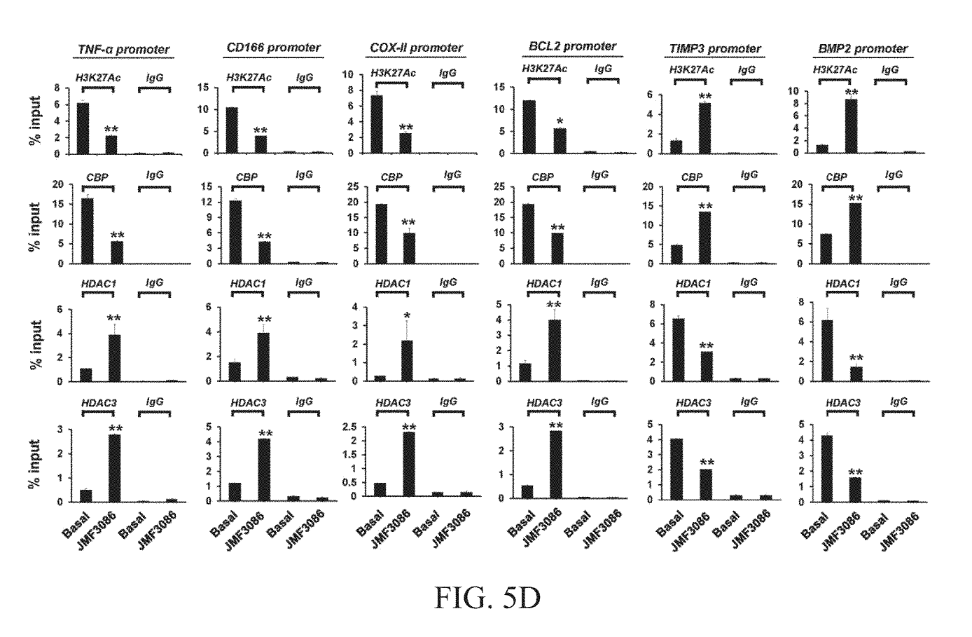

CHIP-on-chip analysis revealed that the binding of H3K27-acetylation to more than 10,000 genes has been altered by JMF3086. Those with two-fold down-regulation or up-regulation in log-ratio were selected and analyzed by Gene Ontology (GO) which categorized them into different biological functions, and the most significant ones were apoptosis and inflammation. Ingenuity Pathways Analysis (IPA) analysis predicted their regulation by NR3C1 and NF-κB, respectively (FIGS. 5A and 5B). JMF3086 inhibited the binding of H3K27-Ac to the gene promoters of inflammation and proliferation, stemness of cancer, and anti-apoptosis as well as reduced their mRNA expression. On the other hand, JM3086 increased the binding of H3K27-Ac to the gene promoters of tumor suppressors and also increased their mRNA expression (FIG. 5C).

To further investigate these differential effects of JMF3086, the bindings of acetyl-H3K27, CBP, HDAC1 and HDAC3 to promoter were examined by quantitative ChIP (qChIP) assays. JMF3086 reduced the bindings of acetyl-H3K27 and CBP to the promoters of TNF-α, CD166, COX-2, BCL2, CXCL1, CXCL2, EpCAM, CD44, Cyclin D1 and MCL1, but increased those of HDAC1 and HDAC3 to the promoters (FIG. 5D). In contrast, JMF3086 increased the bindings of acetyl-H3K27 and CBP to the promoters of TIMP3, BMP2 and p53 but decreased those of HDAC1 and HDAC3 to their promoters (FIG. 5D).

While some of the embodiments of the present invention have been described in detail in the above, it is, however, possible for those of ordinary skill in the art to make various modifications and changes to the particular embodiments shown without substantially departing from the teaching and advantages of the present invention. Such modifications and changes are encompassed in the spirit and scope of the present invention as set forth in the appended claim.

The references listed below and PCT Patent Publication No. WO 2014/015235 cited in the application are each incorporated by reference as if they were incorporated individually.

### REFERENCES

1. Yothers G, et al. (2011) Oxaliplatin as adjuvant therapy for colon cancer: updated results of NSABP C-07 trial, including survival and subset analyses. J Clin Oncol 29(28):3768-3774.
2. Andre T, et al. (2009) Improved overall survival with oxaliplatin, 5-fluorouracil, and leucovorin as adjuvant treatment in stage II or III colon cancer in the MOSAIC trial. J Clin Oncol 27(19):3109-3116.
3. Meyerhardt J A & Mayer R J (2005) Systemic therapy for colorectal cancer. N Engl J Med 352(5):476-487.
4. Minder C M, et al. (2012) Evidence-based use of statins for primary prevention of cardiovascular disease. Am J Med 125(5):440-446.
5. Hawk E & Viner J L (2005) Statins and cancer—beyond the “one drug, one disease” model. N Engl J Med 352(21):2238-2239.
6. Lin Y C, et al. (2008) Statins increase p21 through inhibition of histone deacetylase activity and release of promoter-associated HDAC1/2. Cancer Res 68(7):2375-2383.
7. West A C & Johnstone R W (2014) New and emerging HDAC inhibitors for cancer treatment. J Clin Invest 124(1):30-39.
8. Chen J B, et al. (2013) Design and synthesis of dual-action inhibitors targeting histone deacetylases and 3-hydroxy-3-methylglutaryl coenzyme A reductase for cancer treatment. J Med Chem 56(9):3645-3655.
9. Villeneuve P J & Sundaresan R S (2009) Surgical management of colorectal lung metastasis. Clin Colon Rectal Surg 22(4):233-241.
10. Edwards M S, Chadda S D, Zhao Z, Barber B L, & Sykes D P (2012) A systematic review of treatment guidelines for metastatic colorectal cancer. Colorectal Dis 14(2):e31-47.
11. Terzic J, Grivennikov S, Karin E, & Karin M (2010) Inflammation and colon cancer. Gastroenterology 138(6):2101-2114 e2105.
12. Ullman T A & Itzkowitz S H (2011) Intestinal inflammation and cancer. Gastroenterology 140(6):1807-1816.
13. Greten F R, et al. (2004) IKKbeta links inflammation and tumorigenesis in a mouse model of colitis-associated cancer. Cell 118(3):285-296.
14. Chou C W, Wu M S, Huang W C, & Chen C C (2011) HDAC inhibition decreases the expression of EGFR in colorectal cancer cells. PLoS One 6(3):e18087.

1. A method for treating cancer comprising administering to a subject in need thereof a therapeutically effective amount of a compound represented by formula (I): 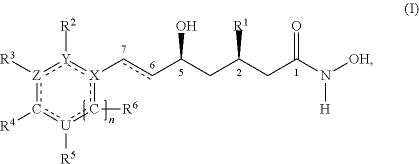
or a pharmaceutically acceptable salt thereof and
a chemotherapeutic agent;
wherein:
n is 0 or 1; [symbol]
represents a single or double bond;
X is carbon;
Y, Z, and U are independently carbon or nitrogen, provided that when Y is carbon and both Z and U are nitrogen, the bond between C6 and C7 is a double bond;
R¹, R², R³, R⁴, R⁵, and R⁶ are independently selected from the group consisting of null, H, C₁₋₆ alkyl, alkenyl, alkynyl, fluoroalkyl, chloroalkyl, bromoalkyl, iodoalkyl, perfluoroalkyl, aryl, heteroaryl, cycloalkyl, cycloalkenyl, aralkyl, aralkenyl, aralkynyl, heteroaralkyl, heteroaralkenyl, heteroaralkynyl, heterocyclyl, acyl, aminocarbonyl, amino, hydroxyl, alkoxy, acyloxy, silyloxy, amido, carbamoyl, and sulfonamido; and
R³ is optionally connected with R² or R⁴ to form carbocycle or heterocycle.

2. The method of claim 1, wherein the chemotherapeutic agent is selected from the group consisting of an alkylating agent, a topoisomerase inhibitor, an antimetabolite agent, an anti-mitotic agent, and any combination thereof.

3. The method of claim 2, wherein the alkylating agent is selected from the group consisting of cyclophosphamide, ifosfamide, cisplatin, carboplatin, oxaliplatin, temozolomide, and any combination thereof.

4. The method of claim 2, wherein the topoisomerase inhibitor is selected from the group consisting of amonafide, amrubicin, amsacrine, campathecin, doxorubicin, epirubicin, etoposide, exatecan, irinotecan, and any combination thereof.

5. The method of claim 2, wherein the antimetabolite agent is selected from the group consisting of 5-fluorouracil, leucovorin, cladribine, capecitabine, mercaptopurine, pemetrexed, methotrexate, gemcitabine, hydroxyurea, cytarabine, and any combination thereof.

6. The method of claim 2, wherein the anti-mitotic agent is selected from the group consisting of paclitaxel, docetaxel, vinblastine, vincristine, vinorelbine, and any combination thereof.

7. The method of claim 1, wherein the subject is a human or a non-human mammal.

8. The method of claim 1, wherein the cancer is caused by proliferation of a neoplastic cell.

9. The method of claim 1, wherein the cancer is selected from the group consisting of a solid tumor, a neoplasm, a carcinoma, a sarcoma, a leukemia, a lymphoma, colorectal cancer (CRC), Hodgkin's lymphoma, Non-Hodgkin's lymphomas, Ewing's sarcoma, multiple myeloma, Wilms' tumor, bone tumors, neuroblastoma, retinoblastoma, testicular cancer, thyroid cancer, prostate cancer, larynx cancer, cervical cancer, nasopharynx cancer, breast cancer, pancreatic cancer, head and neck cancer, esophageal cancer, small-cell lung cancer, non-small-cell lung cancer, brain cancer, melanoma and other skin cancers, a central nervous system (CNS) neoplasm, metastatic CRC, liver metastasis from CRC, lung metastasis from CRC, and any combination thereof.

10. The method of claim 1, further comprising administering at least one additional cancer therapy to the subject.

11. The method of claim 10, wherein the additional cancer therapy is selected from the group consisting of a chemotherapy, a radiation therapy, a targeted therapy, and any combination thereof.

12. The method of claim 11, wherein the targeted therapy comprises administering to the subject a therapeutic agent selected from the group consisting of bevacizumab, trastuzumab and cetuximab.

13. The method of claim 1, wherein the compound of formula (I) sensitizes the cancer cells to the chemotherapeutic agent.

14. The method of claim 1, wherein the compound of formula (I) is 3,5,N-trihydroxy-alkanamide, 3,5,N-trihydroxy-6-alkenamide or a derivative thereof.

15. The method of claim 1, wherein the compound of formula (I) is (3R,5R)-7-{(1S,2S,6R,8S,8aR)-hexahydro-2,6-dimethyl-8-[2-methylbutyryloxy]naphthalenyl}-3,5-dihydroxy-N-hydroxyheptanamide.

16. The method of claim 1, wherein the compound of formula (I) and the chemotherapeutic agent are administered concurrently, separately or sequentially.

17. The method of claim 1, wherein after the administrating step, a gene promoter associated with inflammation, proliferation, stemness of cancer, or anti-apoptosis is down-regulated.

18. The method of claim 17, wherein the gene promoter associated with inflammation, proliferation, stemness of cancer, or anti-apoptosis is selected from the group consisting of TNF-α, CXCL1, CXCL2, COX-II, Cyclin D1, CD166, EpCAM, CD44, BCL2, MCL1, and any combination thereof.

19. The method of claim 1, wherein after the administrating step, a gene promoter of a tumor suppressor is up-regulated.

20. The method of claim 19, wherein the gene promoter of the tumor suppressor is selected from the group consisting of TIMP3, BMP2, p53, and any combination thereof.

21. A method for treating metastatic colorectal cancer (CRC) comprising administering to a subject in need thereof a therapeutically effective amount of a compound represented by formula (I): 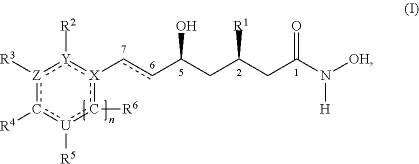
or a pharmaceutically acceptable salt thereof;
wherein:
n is 0 or 1; [symbol]
represents a single or double bond;
X is carbon;
Y, Z, and U are independently carbon or nitrogen, provided that when Y is carbon and both Z and U are nitrogen, the bond between C6 and C7 is a double bond;
R¹, R², R³, R⁴, R⁵, and R⁶ are independently selected from the group consisting of null, H, C₁₋₆ alkyl, alkenyl, alkynyl, fluoroalkyl, chloroalkyl, bromoalkyl, iodoalkyl, perfluoroalkyl, aryl, heteroaryl, cycloalkyl, cycloalkenyl, aralkyl, aralkenyl, aralkynyl, heteroaralkyl, heteroaralkenyl, heteroaralkynyl, heterocyclyl, acyl, aminocarbonyl, amino, hydroxyl, alkoxy, acyloxy, silyloxy, amido, carbamoyl, and sulfonamido; and
R³ is optionally connected with R² or R⁴ to form carbocycle or heterocycle.

22. The method of claim 21, wherein the metastatic CRC is selected from the group consisting of liver metastasis from CRC, lung metastasis from CRC, and any combination thereof.

23. The method of claim 21, further comprising administering at least one additional cancer therapy to the subject.

24. The method of claim 23, wherein the additional cancer therapy is selected from the group consisting of a chemotherapy, a radiation therapy, a targeted therapy, and any combination thereof.

25. The method of claim 24, wherein the targeted therapy comprises administering to the subject a therapeutic agent selected from the group consisting of bevacizumab, trastuzumab and cetuximab.

26. The method of claim 21, wherein the compound of formula (I) is 3,5,N-trihydroxy-alkanamide, 3,5,N-trihydroxy-6-alkenamide or a derivative thereof.

27. The method of claim 21, wherein the compound of formula (I) is (3R,5R)-7-{(1S,2S,6R,8S,8aR)-hexahydro-2,6-dimethyl-8-[2-methylbutyryloxy]naphthalenyl}-3,5-dihydroxy-N-hydroxyheptanamide.

28. The method of claim 21, wherein after the administrating step, a gene promoter associated with inflammation, proliferation, stemness of cancer, or anti-apoptosis is down-regulated.

29. The method of claim 28, wherein the gene promoter associated with inflammation, proliferation, stemness of cancer, or anti-apoptosis is selected from the group consisting of TNF-α, CXCL1, CXCL2, COX-II, Cyclin D1, CD166, EpCAM, CD44, BCL2, MCL1, and any combination thereof.

30. The method of claim 21, wherein after the administrating step, a gene promoter of a tumor suppressor is up-regulated.

31. The method of claim 30, wherein the gene promoter of the tumor suppressor is selected from the group consisting of TIMP3, BMP2, p53, and any combination thereof.

32. A kit comprising a therapeutically effective amount of a compound represented by formula (I): 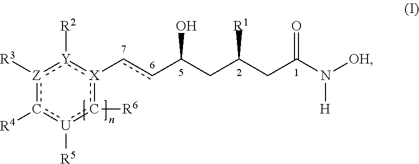
or a pharmaceutically acceptable salt thereof and
a chemotherapeutic agent;
wherein:
n is 0 or 1; [symbol]
represents a single or double bond;
X is carbon;
Y, Z, and U are independently carbon or nitrogen, provided that when Y is carbon and both Z and U are nitrogen, the bond between C6 and C7 is a double bond;
R¹, R², R³, R⁴, R⁵, and R⁶ are independently selected from the group consisting of null, H, C₁₋₆ alkyl, alkenyl, alkynyl, fluoroalkyl, chloroalkyl, bromoalkyl, iodoalkyl, perfluoroalkyl, aryl, heteroaryl, cycloalkyl, cycloalkenyl, aralkyl, aralkenyl, aralkynyl, heteroaralkyl, heteroaralkenyl, heteroaralkynyl, heterocyclyl, acyl, aminocarbonyl, amino, hydroxyl, alkoxy, acyloxy, silyloxy, amido, carbamoyl, and sulfonamido; and
R³ is optionally connected with R² or R⁴ to form carbocycle or heterocycle.

33. The kit of claim 32, wherein the compound of formula (I) is selected from the group consisting of 3,5,N-trihydroxy-alkanamide, 3,5,N-trihydroxy-6-alkenamide and a derivative thereof.

34. The kit of claim 32, wherein the compound of formula (I) is (3R,5R)-7-{(1S,2S,6R,8S,8aR)-hexahydro-2,6-dimethyl-8-[2-methylbutyryloxy]naphthalenyl}-3,5-dihydroxy-N-hydroxyheptanamide.

35. The kit of claim 32, wherein the chemotherapeutic agent is selected from the group consisting of an alkylating agent, a topoisomerase inhibitor, an antimetabolite agent, an anti-mitotic agent, and any combination thereof.

36. The kit of claim 35, wherein the alkylating agent is selected from the group consisting of cyclophosphamide, ifosfamide, cisplatin, carboplatin, oxaliplatin, temozolomide, and any combination thereof.

37. The kit of claim 35, wherein the topoisomerase inhibitor is selected from the group consisting of amonafide, amrubicin, amsacrine, campathecin, doxorubicin, epirubicin, etoposide, exatecan, irinotecan, and any combination thereof.

38. The kit of claim 35, wherein the antimetabolite agent is selected from the group consisting of 5-fluorouracil, leucovorin, cladribine, capecitabine, mercaptopurine, pemetrexed, methotrexate, gemcitabine, hydroxyurea, cytarabine, and any combination thereof.

39. The kit of claim 35, wherein the anti-mitotic agent is selected from the group consisting of paclitaxel, docetaxel, vinblastine, vincristine, vinorelbine, and any combination thereof.

40. The kit of claim 32, further comprising a therapeutic agent for a targeted therapy.

41. The kit of claim 40, wherein the therapeutic agent is bevacizumab, trastuzumab or cetuximab.
